(19) United States
(12) Patent Application Publication
(10) Pub. No.: US 2021/0068753 A1
(43) Pub. Date: Mar. 11, 2021

(54) IMAGING ELECTRICAL CURRENT PATTERNS GENERATED BY A MEDICAL DEVICE

(71) Applicant: ARZONA BOARD OF REGENTS ON BEHALF OF THE UNIVERSITY OF ARIZONA, Tucson, AZ (US)

(72) Inventors: Chet Preston, Tucson, AZ (US); Yexian Qin, Tucson, AZ (US); Charles Ingram, Tucson, AZ (US); Alex Burton, Tucson, AZ (US); Russell S. Witte, Tucson, AZ (US)

(21) Appl. No.: 16/644,853

(22) PCT Filed: Sep. 7, 2018
(86) PCT No.: PCT/US2018/049947

§ 371 Date: Mar. 5, 2020

Related U.S. Application Data
(60) Provisional application No. 62/555,524, filed on Sep. 7, 2017.

(51) Int. Cl. A61B 5/00; A61B 5/06

(52) U.S. Cl. CPC A61B 5/686; A61B 5/0093; A61B 5/065; A61B 5/4836; A61B 5/0042; A61B 5/4064

(57) ABSTRACT

Acoustoelectric imaging (AEI) offers a novel, non-invasive method for monitoring current densities produced by a deep brain stimulator (DB S) or other medical device. By providing visual feedback of the electrical current patterns produced by the device, AEI may help guide placement of an implant during surgery, monitor device performance during routine checkups and over time, and perform accurate calibrations (in vivo or in situ).

### CROSS REFERENCE TO RELATED APPLICATION(S)

This application claims priority to U.S. provisional patent application No. 62/555,524, filed on Sep. 7, 2017, which is hereby incorporated herein by reference in its entirety.

### GOVERNMENT SPONSORSHIP

This invention was made with government support under Grant No. R24 MH109060 awarded by NIH. The government has certain rights in the invention.

### FIELD OF THE INVENTION

Embodiments are in the field of systems and methods for imaging. More particularly, embodiments disclosed herein relate to systems and methods for acoustoelectrically imaging time-varying current densities in the brain or other body parts.

### BACKGROUND OF THE INVENTION

Deep brain stimulation (DBS) is an effective treatment for motor symptoms resulting from Parkinson's disease (PD), essential tremor and dystonia. This success encouraged further investigations into the use of DBS as a treatment for other neurological disorders, including epilepsy, depression, Tourette's syndrome and obsessive compulsive disorder. In PD, DBS appears to work by normalizing pathological low-frequency oscillations in the basal ganglia and basal ganglia cortical circuits, but the exact mechanisms underlying therapeutic DBS remain unknown. Regardless, success for DBS strongly depends on the accurate placement of DBS electrodes in the subthalamic nucleus or globus pallidus interna. Although computed tomography and magnetic resonance imaging are commonly used to help guide placement during surgery, these techniques are unable to directly visualize the contacts or map current patterns for real-time feedback during surgery. Computational models are also employed for pre-surgical planning to predict current spread in the brain and optimal placement of the leads. These models, however, are primarily theoretical and lack valuable empirical in vivo data for validation and optimization.

Thus, it is desirable to provide embodiments of a system and method for acoustoelectrically imaging time-varying current densities in the brain or other body parts that do not suffer from the above drawbacks.

These and other advantages of the present invention will become more fully apparent from the detailed description of the invention herein below.

### SUMMARY OF THE INVENTION

Embodiments are directed to a method for acoustoelectrically imaging, within a body part, time-varying current densities generated by a medical device. In an embodiment, the method comprises: generating time-varying current densities with a medical device; applying a sound beam within 0-10 cm from the medical device to generate acoustoelectric (AE) interaction signals proportional to the time-varying current densities; detecting the AE interaction signals using one or more recording electrode; and imaging the time-varying current densities using the detected AE interaction signals. Embodiments of the method are capable of acoustoelectrically imaging time-varying current densities in the brain or other body parts.

Embodiments are also directed to an AE imaging system that acoustoelectrically images, within a body part, time-varying current densities generated by a medical device. In an embodiment, the AE imaging system comprises: a medical device that generates time-varying current densities; a sound beam system that applies a sound beam within 0-10 cm from the medical device to generate AE interaction signals proportional to the time-varying current densities; one or more recording electrode that detects the AE interaction signals; and a current density imaging system that images the time-varying current densities using the detected AE interaction signals. Embodiments of the system are capable of acoustoelectrically imaging time-varying current densities in the brain or other body parts.

Additional embodiments and additional features of embodiments for the method for acoustoelectrically imaging and AE imaging system are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It's understood, however, that the inventive concepts disclosed herein are not limited to the precise arrangements and instrumentalities shown in the figures. The detailed description will refer to the following drawings in which like numerals, where present, refer to like items.

FIG. 1A is a schematic diagram illustrating a 1 MHz ultrasound transducer submerged in water focused on a container filled with physiologic saline. A DBS implant is oriented perpendicular to the ultrasound beam. FIG. 1B illustrates a DBS implant with an ultrasound transducer below. DBS contacts are labeled “+”, “−”, or “R” signifying the source or sink for stimulation or the recording electrode, respectively.

FIGS. 2A-2C are diagrams illustrating a time varying stimulation with an M-Mode AE signal. FIG. 2A depicts the AE signal (highlighted by a bracket) with a 1 MHz carrier frequency associated with the ultrasound transducer occurring at the depth of the electrode.

FIG. 2B illustrates an injected current burst waveform from the DBS contacts measured across a 1 Ohm resistor. FIG. 2C illustrates the resulting baseband M-mode AE signal from the four rectangular pulses on a dB scale with a dynamic range of ±6 dB from peak signal. The bottom and top pulses represent source and sink current densities, respectively.

FIG. 3A is a diagram illustrating a plot of SNR vs current density. The vertical dashed line indicates 3V stimulation. FIG. 3B is a diagram illustrating a plot of sensitivity (slope) of the AE signal calculated by the quotient of the AE amplitude (mV) per current density times (mA/cm²) given ultrasound induced acoustic pressure of 3.44 MPa. Each point indicates the mean and standard deviation of 4 pulse sequences.

FIGS. 4A and 4B are diagrams illustrating B-mode images of AE and PE signals. The figures are images of the DBS implant oriented parallel and perpendicular to the ultrasound beam, respectively. The AE signals of current densities overlay the PE image in hot (source) and cold (sink) colors. Brightness of color depicts magnitude. A center point on the Y axis was used when generating the X, Z B-mode image, and vice-versa. FIG. 4C illustrates a table comparing the physical dimensions of the DBS contacts with the AE signals surrounding the contacts during stimulation. The distance between the source and sink contacts was estimated using both the parallel (Ax) and perpendicular (Lat) orientations.

FIGS. 5A and 5B are diagrams illustrating a timing diagram and an experimental setup, respectively. FIG. 5A illustrates a timing diagram with AE and pulse-echo (PE) ultrasound acquisition. An event trigger initiates a burst of electrical stimulation pulses (3 V, 1-ms pulse, 200 Hz) and ultrasound pulses (Dt=250 ms). Each ultrasound pulse was used to generate an AE signal along the depth axis (A-line) detected on the recording electrode. The amplitude of the AE signal was proportional to the local and instantaneous current densities generated by the deep brain stimulation device (lower left, displaying a monopole at contact 3). The backscattered echo was also detected by the ultrasound transducer to form standard pulse echo images co-registered with AE imaging. FIG. 5B is a diagram illustrating a side view of an exemplary setup with a wiring diagram for both monopole (dashed) and dipole (solid) stimulating configurations. The ultrasound transducer is mechanically steered along the x-axis to form 3-D data sets (X, Z, t_{slow}). Experiments were performed with and without the human skullcap.

FIGS. 6A-6E are diagrams illustrating AE M-mode images of time-varying current. FIG. 6A illustrates a plot of a darker trace which is a measured current waveform injected between contacts 0 and 3 of the deep brain stimulation implant. The lighter trace is a waveform of AE(t) at a depth of 67.5 mm (indicated by white dashed line in FIG. 6B. Amplitude of the lighter trace is normalized to the amplitude of the measured current to highlight the similarities in the waveform; y-axis labels refer to the darker trace in milli-amperes. FIGS. 6B-6E illustrate corresponding AE M-mode images related to the time-varying injected current in FIG. 6A, indicating raw AE amplitude signals (FIG. 6B), AE amplitude signal filtered in slow (t_{slow}) and fast (t_{fast}) time (FIG. 6C), magnitude of Hilbert-transformed signal in FIG. 6B with 6-dB dynamic range (FIG. 6D) and signed magnitude of the basebanded and filtered signal indicating polarity (color) and amplitude (intensity) of injected current at the anode with ±6-dB dynamic range (FIG. 6E). The ultrasound transducer is located at a depth of 0 mm. Vertical dotted arrows from FIG. 6A to FIG. 6B indicate the peaks of the injected current pulses, which correspond well to the positive peaks of the AE signals located at the depth of the deep brain stimulation implant (˜67.5 mm from the ultrasound transducer).

FIGS. 7A and 7B are diagrams illustrating AE images of deep brain stimulation (DBS) monopoles in saline. FIG. 7A illustrates two-dimensional cross-sectional images of the AE signals during monopole stimulation patterns at peak current (t=5.5 ms). Gray represents co-registered PE image of the DBS device at a depth of 67.5 mm. Hot colors represent the magnitude of the AE signal. Below each image is a sized-to-scale depiction of the DBS device with the contact used to generate the monopole indicated by a circle. FIG. 7B is a diagram illustrating a plot of the envelope of the AE signal along the long axis of the DBS device for each monopole configuration. Vertical dashed lines indicate a center of contact used for stimulation.

FIGS. 8A and 8B are diagrams illustrating AE images of deep brain stimulation (DBS) dipoles in saline. FIG. 8A illustrates a two-dimensional AE imaging of dipoles generated using the clinical DBS device at peak current (t_{slow}=5.5 ms). Gray indicates pulse echo image. Color indicates the polarity and magnitude of the AE signal; hot and cold colors describe positive and negative voltages. The DBS device is depicted to scale below each image, with a right-side circle and left-side circle indicating the anode and cathode, respectively. FIG. 8B is a diagram illustrating a plot comparing the lateral position of the AE imaging peaks with the center location of the contacts used as the cathode and anode. AE peaks and contact locations are displayed relative to the center of contact 0 (determined by pulse echo). Contact 3 was used as the anode for the dipole configuration. Circles are labeled with numbers corresponding to the contacts used in the different dipole configurations. The slope of the dashed line is determined from the actual distance from contact 0.

FIGS. 9A-9C are diagrams illustrating plots of AE sensitivity and current detection thresholds. FIG. 9A illustrates a waveform of the injected current (black) overlaid with the measured AE signal from a monopole (gray). FIG. 9B illustrates AE sensitivity per unit pressure using a monopole generated by a deep brain stimulation contact (averages=20).

FIG. 9C illustrates a plot of signal-to-noise ratio (SNR) versus number of averages for measuring the AE signal. The solid line represents the best fit through the data (2.74 dB/octave). The dotted line represents the expected slope for completely independent observations (3 dB/octave).

FIGS. 10A-10D are diagrams illustrating calibration of an ultrasound beam through a human skullcap. FIG. 10A illustrates a human skullcap used for transcranial AE imaging experiments. The dashed circle and arrow denote a region for delivering ultrasound. FIGS. 10B and 10C are diagrams illustrating plots of a time waveform and frequency spectrums of ultrasound pulses recorded by the hydrophone. FIG. 10D illustrate lateral pressure profiles of an ultrasound beam at focus with and without the skullcap.

FIGS. 11A and 11B are diagrams illustrating AE imaging of deep brain stimulation (DBS) monopoles and dipole through a human skull. FIG. 11A illustrates a B-Mode pulse echo image of the DBS electrode through the skull (gray) superimposed on the AE signal from a dipole at peak current generated between contacts 0 and 3. The dashed box between 60 and 68 mm indicates the zoomed region of interest for the images depicted in FIG. 11B. FIG. 11B (Top four images) illustrates two-dimensional AE images of monopoles through a skull with a dynamic range of 6 dB; FIG. 11B (bottom) illustrates a 2-D AE image of a dipole (±6 dB) superimposed on the pulse echo image (gray) of the DBS electrode. The right-side circle and the left-side circle (in the bottom image of FIG. 11B) of the DBS electrode indicate the contacts used as the anode and cathode, respectively. The circles in the top 4 images in FIG. 11B only indicate an anode.

FIG. 12 is a flowchart illustrating an embodiment of a method for acoustoelectrically imaging, within a body part, time-varying current densities generated by a medical device, in accordance with an embodiment.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical AE imaging system and typical method for acoustoelectrically imaging. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

There are potentially many different target areas/applications for the method/system described in this disclosure, although the brain is the focus herein for purposes of explanation only.

There are no techniques to the inventors' knowledge that are routinely used to image the current pattern (in 3D or 4D) near a medical stimulator. Performance is typically determined by electrical impedance measurements or other indirect techniques that do not provide much, if any, spatial information. Electrical impedance imaging does not typically have adequate spatial resolution.

Acoustoelectric imaging (AEI) offers a novel, non-invasive method for monitoring current densities produced by a deep brain stimulator (DBS) or other medical device. By providing visual feedback of the electrical current patterns produced by the device, AEI may help guide placement of an implant during surgery, monitor device performance during routine checkups and over time, and perform accurate calibrations of the DBS or medical device (in vivo or in situ).

Electrical stimulation is a common technique for treating a variety of medical conditions ranging from lower back pain to essential tremor caused by Parkinson's Disease. Medical devices, such as the Transcutaneous Electrical Nerve Stimulator (TENS) and DBS, rely on the accurate delivery of electrical current to the afflicted region. Because the therapeutic effect from these devices often depends on the pattern of stimulation (in space and time), visual feedback is highly desirable to not only calibrate the device and ensure proper operation, but to also optimize performance during therapy. Thus, this invention describes AEI as a new technique to remotely map current density patterns produced by a medical device or implant. AEI is based on the acoustoelectric (AE) effect, an interaction between a focused ultrasound beam and a material's resistivity to map current source densities in 3D. AEI is real-time, non-invasive, and provides high spatial resolution determined by the ultrasound wavelength (˜1 mm). The method is co-registered with standard ultrasound imaging that describes structure and anatomy with current density maps (AEI). This disclosure describes AE imaging of current source density patterns produced by, for example, a commercial DBS implant (Medtronic #3389). AEI is capable of rapidly visualizing current densities, direction and location produced by the DBS device. The technique of AEI can be applied to other types of medical devices or implants that use electrical or magnetic stimulation, such as transcranial magnetic stimulation (TMS), cardiac pacemakers, transcutaneous electrical nerve stimulation (TENS) or vagal nerve stimulators. As a tool for non-invasive monitoring of electrical stimulation, AEI has the potential to enhance performance of existing medical devices and improve care for patients suffering from a variety of debilitating and costly medical conditions.

### 1. Acoustoelectric Imaging of Time-Varying Current Produced by a Clinical Deep Brain Stimulator

DBS is an effective treatment for a variety of brain disorders, including Parkinson's disease, depression, Tourette's and chronic pain. However, there is no reliable method to non-invasively image electric current flow generated by a DBS. The inventors employ 4D current source density imaging based on the AE effect, which integrates an ultrasound beam with electrical recording, to map current flow produced by a clinical DBS device. AE imaging was able to accurately determine the polarity, magnitude and location of the current densities near the DBS device placed in physiologic saline with a signal-to-noise ratio of 17.1 dB using stimulation parameters similar to what are used on patients. Pulse echo (PE) ultrasound was acquired simultaneously to provide additional information regarding the spatial coordinates and structure of the DBS without need of additional techniques. These results suggest that AE imaging combined with PE ultrasound may provide valuable feedback during and after implantation of a DBS device.

1.1. Theory

DBS is an effective treatment for some movement and psychological disorders. It is most commonly known for its application in minimizing tremors resulting from prolonged use of Levodopa in treating Parkinson's disease (PD). Ultimately, DBS appears to enact its effect by regularizing pathological low frequency oscillations in the basal ganglia. However, the entirety of the neural circuitry excited via DBS remains unknown. One avenue for further determining how DBS facilitates symptomatic relief through specific excitation of the basal ganglia is to determine the spatiotemporal characteristics of the current densities generated by the implant. In this disclosure, the inventors employ AEI as a new technique to map time-varying current produced by a DBS.

AEI relies on fluctuations in current densities induced by the AE effect during the propagation of an acoustic wave, which modulates tissue resistivity as a function of pressure described as,

$$\frac{Δρ}{{ρ}_{0}} = - K Δ P \qquad ( 1 )$$

where ρ₀ is the initial electrical resistivity of the tissue, is the change in tissue resistivity due to acoustic pressure ΔP, and K is an AE interaction constant on the order of 0.1%/Mpa in biological tissue. The spatial and temporal resolutions for AEI is defined by the ultrasound transducer and beam shape, typically in the sub-millimeter and sub-millisecond ranges. The resulting AE signal detected on an electrode is proportional to the local current density and pressure of the acoustic wave, which is maximum within the focal zone of the ultrasound transducer, given by

$${V}_{AE} ( x , y , z , t ) = \int \int \int ( {J}_{L} \cdot {J}_{I} ) ( - {K}_{I} {ρ}_{0} {P}_{0} a ( t - \frac{z}{c} ) ) dxdydz \qquad ( 2 )$$

where J_{L}·J_{I} is the inner product between the current densities J_{I} and lead field J_{L}, and

$$Δ P ( t ) = {P}_{0} a ( t - \frac{z}{c} ) \qquad ( 3 )$$

is the time varying change in pressure within the spatial bounds defined by the ultrasound transducer at time t. AEI has been used to detect current densities generated from dipoles as well as the cardiac depolarization wave in the live rabbit heart.

A goal of this disclosure is to assess baseline performance of AE technology for detecting and resolving current densities near a DBS implant using clinically-relevant stimulation parameters. More specifically, the inventors investigated the effectiveness of AEI in resolving the current directionality and density distributions. The inventors also discuss potential applications for the new technology to provide feedback during and after a DBS implantation surgery.

1.2. Methods

A. Experimental Setup

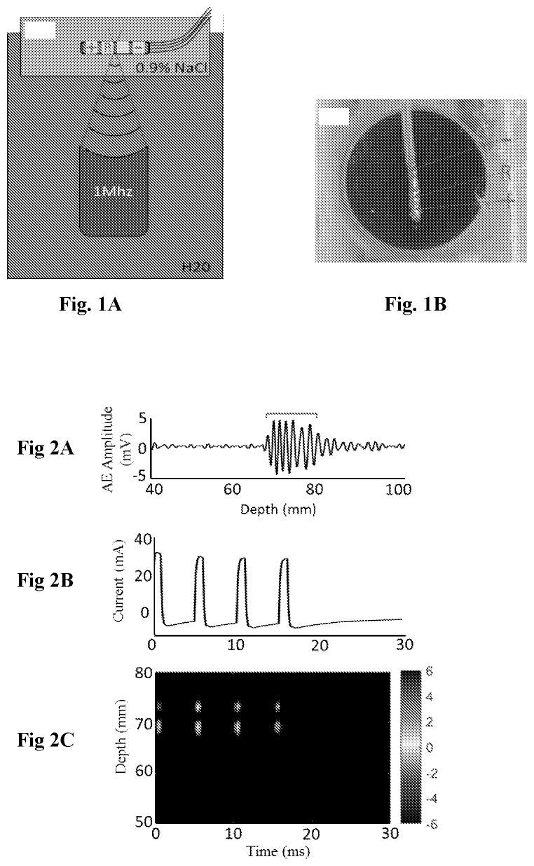

A commercial DBS implant (Medtronic #3389) with four platinum-iridium electrodes was placed in a bath of 0.9% NaCl, as illustrated in FIGS. 1A and 1B. The linear array of contacts on the implant were positioned either parallel or perpendicular to the propagation of the ultrasound beam to mimic either burr hole or temporal window ultrasound placement, respectively. A single element transducer (1 MHz, Olympus NDT A392S, 3.44 MPa at focal length 63 mm) produced a focused ultrasound beam at 4 kHz repetition rate in the region of the DBS, while current was passed between two of its leads. The depth and stimulating parameters were chosen to mimic typical clinical DBS parameters.

B. Current Generation and Data Acquisition

Time-varying current (4 square pulses, 1 msec duration, 5 msec inter pulse interval) was generated (Agilent 33220A) by applying pulses up to 6V between two electrodes (shown with + and − in FIGS. 1A and 1B). Injected current was modulated by the ultrasound beams at a pulse repetition frequency of 4000 Hz. The current was measured across a 1 Ohm resistor. The AE signal was recorded on a contact situated between the stimulating sites and sampled at 20 MHz. The AE interaction signal was captured using a custom pre-amplifier and digital acquisition card (NI PXI-5105). The ultrasound transducer was mechanically scanned in the lateral plane to form 4D AE images related to the location, direction and amplitude of the current densities. The AE signal was averaged 50 times at each point and amplified 200× during the acquisition of 4D data. Stimulation amplitude of 3V was used for experiments when the voltage was fixed. Standard pulse echo ultrasound was also captured simultaneously on an Olympus 5077 pulse receiver to identify the position of the DBS implant.

C. Signal Processing and Analysis

Raw AE signals were band-pass filtered between 0.5 and 1.5 MHz in ultrasound time (i.e., “fast” time) and between 100 and 1800 Hz in physiologic time (i.e., “slow” time). AE signals were demodulated to baseband frequencies to determine the direction of current flow from the sign of the baseband signal. The signal-to-noise ratio (SNR) at varying current densities was calculated using the root mean squared (RMS) value of the AE signal compared to the RMS of an equal duration section devoid of current. Sensitivity (μV/(MPa*mA/cm²)) was calculated using the peak AE signal divided by the product of the pressure of the ultrasound waves (3.44 MPa), and the current per lateral area determined by the focal width of the ultrasound transducer (1.72 mm²). Spatial extent of each pole of the dipole generated by the DBS was calculated at full-width half-maximum (FWHM) of the B mode images based on the magnitude of the AE signals. The dimensions of the DBS contacts were estimated from the AE B mode images with consideration of the ultrasound focal size.

1.3. Results

The time varying pulse sequence with resulting baseband M-mode image of DBS implant oriented parallel to the ultrasound beam is shown in FIGS. 2A-2C. The positive rectangular pulses (FIG. 2B) resulted in the AE signals presented in FIG. 2A at a depth of 68-76 mm from the ultrasound transducer. The M-mode image is presented in FIG. 2C, which uses the sign of the baseband AE signal (demodulated at 1 MHZ) to determine the polarity of the current densities. The right-side shade bar in ±dB clearly indicates the polarity of the dipole, while the brightness indicates the magnitude.

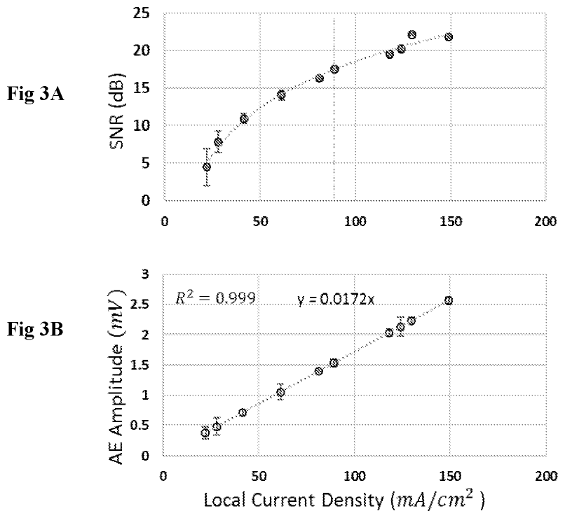

The SNR of the AE signal was above 6 dB for stimulation levels at or above 1V. This corresponded to local current densities of approximately 25 mA/cm² (FIG. 3A). SNR continued to increase logarithmically to −22 dB at local current densities of 140 mA/cm² resulting from 5.5V stimulation. Within the common range of stimulation levels used in the clinic of 3V, SNR was 17.1 dB with 50 averages. AE amplitude increased linearly (R²=0.999) with local current density, resulting in a sensitivity of

$$17.2 \frac{μ V}{MPa \cdot ( mA \text{/} {cm}^{2} )}$$

### at 3.44 MPa (FIG. 3B).

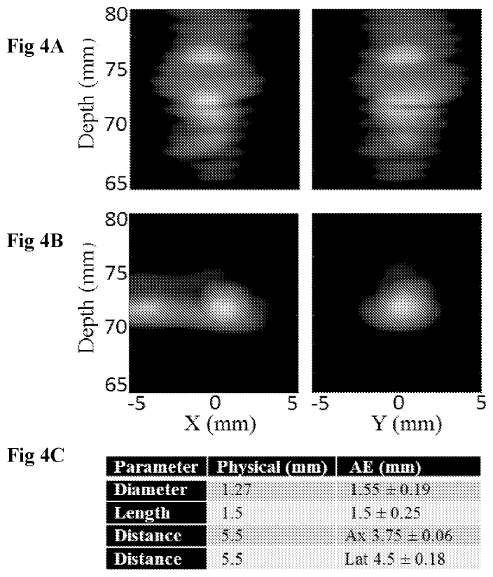

FIGS. 4A and 4B depict the pulse echo (grayscale) of the DBS electrode and the superimposed acoustoelectric signal (hot/cold colors) during the peak of a current pulse for a DBS electrode oriented parallel and perpendicular to the ultrasound beam, respectively. The dimensions of the DBS contacts were measured using the AE signal and compared with their known physical dimensions (FIG. 4C). Mean diameter of the contact using the AE signal was measured to be 1.55 mm. This was 0.28±0.19 mm, larger than the actual diameter of 1.27 mm. Mean length of a contact, on the other hand, matched the physical size of the contact (1.5 mm) with a standard deviation of 0.25 mm. The estimated distance between the centers of the two stimulating contacts had a larger divergence from the physical dimensions than was seen in the estimation of a single contact. While the actual physical distance between the two sites was 5.5 mm, the distance measured via the AE signal was 3.75±0.06 mm in the parallel configuration and 4.5±0.18 mm in the perpendicular configuration.

1.4. Discussion and Conclusion

Using AEI, the inventors successfully measured current densities generated by a clinical DBS device. 4D reconstruction of the AE signal gave clear magnitude, location, and relative polarity of the current densities over time. Additionally, simultaneously acquired PE data revealed the electrode position. Using clinically relevant stimulation parameters, the invention achieved an SNR of 17.1 dB. Table 1 below indicates how the stimulation parameters varied from other AE studies to more closely resemble those used in human subjects who have received a DBS implant. Of these parameters, the pulse width diverged most strongly from those used in clinic. However, this was done intentionally to provide additional data points per pulse. Only a single convolution between the ultrasound beam and the electric signal is required to fully reconstruct the AE signal. Therefore, with correctly controlled timing, minimal detectable pulse-width corresponds to the sampling frequency in the depth axis and the size of the local current density to be measured in the depth direction. In an experimental setup, the theoretical minimal pulse length is in the range of a few microseconds. In other words, there is no physical limitation that would restrict the usage of a clinically representative pulse width.

TABLE 1
Experimental vs. standard clinical stimulation parameters
using Medtronic 3387 or 3389 DBS implant

| | | |
|---|---|---|
| Parameter | Experimental | Clinical |

| | | | | |
|---|---|---|---|---|
| Stimulation Intensity | 3 | V | ~3.5 | V |
| Pulse Duration | 1 | ms | ~0.0085 | ms |
| Pulse Frequency | 200 | Hz | ~165 | Hz |

One important reason why current source density imaging is important in optimizing DBS parameters is the introduction of a new clinical implant (the St. Jude's Infinity)—that allows for electric current steering in precise directions. Precise current steering enables better control of the orientation between axons and electric fields. Neural depolarization is dependent on numerous characteristics independent of the stimulating, source such as axon diameter or myelination, but it also depends on the flux of current parallel to its axon. In the case of strong inter-subject structural homogeneity, prior knowledge of the anatomical structure of the basal ganglia can allow, to some extent, electric fields to be designed that maximize the current flux along beneficial axons or to minimize it across detrimental ones.

The two main sites for DBS implantation for relieving dyskinesia are the globus pallidus interna and the subthalamic nucleus. However, although only 7 mm apart, stimulation at each location is associated with different side-effects such as increased depression or impaired visuomotor speed. Given both the variety of complex side-effects associated with DBS and the introduction of current-steerable implants, the use of AEI may greatly aid in the determination of optimal stimulation parameters by refining the spatiotemporal spread of DBS generated current densities.

Spatial resolution of a dipole using AEI is dependent on the bandwidth of the ultrasound transducer. Here, the inventors showed that reasonably accurate spatial properties can be determined via the AE signal using a wavelength as large as 1.48 mm when using step sizes of 0.5 mm in the lateral/elevational plane. Given the relatively large (1.27 mm) diameter of the DBS contacts, resolution of the drop in current densities surrounding the implant can be further increased by decreasing the step sizes to allow for more complete overlapping of the current density volume when using the same ultrasound transducer.

The two major areas of use for ultrasound regarding DBS implants would be during initial implant placement followed by chronic monitoring of the stimulation currents. Current methods use intraoperative fluoroscopy during placement, and computed tomography (CT) or magnetic resonance imaging (MRI) for post-operative monitoring of the implant. Transcranial ultrasound can be used in tandem with these other imaging modalities to provide additional information regarding the current densities generated by the DBS leads.

Overall, this disclosure demonstrates that AEI can accurately provide location, density, and directionality of current generated by a clinical DBS device without the inclusion of additional electrodes. These results make realistic the prospect of non-invasive current source imaging, combined with PE ultrasound, for both initial implant placement and chronic monitoring of changes over time regarding current generation or lead migration.

### 2. Additional Examples and Further Analysis

As discussed above, the inventors employ AEI as a new technique to noninvasively map the location, magnitude and polarity of current source densities generated by a clinical DBS device. AEI exploits an interaction between ultrasound pressure and tissue resistivity to remotely detect and map current densities with high spatial and temporal resolution. As an ultrasound beam is pulsed and swept in a conductive medium, a recording electrode detects the high frequency AE interaction signal, which is proportional to the local pressure and current. Feasibility of AEI for mapping current densities has been demonstrated in a variety of preparations, including time-varying dipoles and imaging of the cardiac depolarization wave in the live rabbit heart. Goals for this application are to 1) assess the performance (spatial resolution, sensitivity, and accuracy) of AEI for detecting and resolving current densities near a DBS device using stimulation parameters resembling those used clinically; and 2) demonstrate feasibility and benchmark performance of AEI through a human skullcap.

2.1 Material and Methods

A. Acoustoelectric Imaging: Theory

The AE effect describes the interaction of an acoustic wave propagating through a conductive medium. As the ultrasound wave propagates, the density of the medium is modulated by the pressure resulting in changes in the medium's resistivity. In accordance with Ohm's law, a voltage can be measured based on the product of this induced change in resistivity with the inner product of the current density (J_{I}(x,y,z,t_{slow})) and a recording lead field from a pair of recording electrodes (J_{L}(x, y, z)) integrated over a volume. With an ultrasound transducer centered at coordinates x₁ and y₁, the recorded voltage of the AE signal, V_{AE}, includes additional terms related to the ultrasound parameters due to the AE effect:

V_{AE}(x₁,y₁,t_{fast},t_{slow})=−K∫∫∫(J_{L}·J_{I})ρ₀b(x−x₁,y−y₁,z)P₀α(t_{fast}−z/c)dxdydz, (4)

with P₀ the initial resistivity, ΔP the varying acoustic pressure, K an acoustoelectric interaction constant (e.g., 0.034 in 0.9% saline), ultrasound beam pattern b(x,y,z), pulse amplitude P₀, and ultrasound pulse waveform a(t_{fast}). The expression includes both fast and slow time components, where fast time (t_{fast}) refers to the propagation of ultrasound waves (μs) with wave velocity c along the z axis, and slow time (t_{slow}) refers to the time frame of the injected current waveform (ms), referring to how the measured current densities, J_{I}, change as the injected current varies over time. The AE signal is further separated from the recorded potential using a high pass filter.

Based on Eq. (4), an AE M-Mode image (z vs. t_{slow}; analogous to M-Mode pulse echo) is formed by recording V_{AE} while producing a sequence of ultrasound pulses. A raster scan of the ultrasound transducer along x and y produces volumetric AE images and 4D movies (volume vs. t_{slow}). Note, the AE signal is confined to the ultrasound beam such that the spatial resolution for AEI depends on the acoustic wavelength (˜1.5 mm at 1 MHz in water) and size of the focus.

B. Experimental Setup

A commercial DBS device (Medtronic model #3389, Medtronic, Inc., Minneapolis, Minn., USA) with four platinum-iridium electrodes was placed in a bath of 0.9% NaCl. The device has 4 symmetric contacts numbered 0-3 beginning at the distal tip. Each contact has a length of 1.5 mm, diameter of 1.27 mm, and separation (kerf) of 0.5 mm. The array of electrodes on the device were positioned perpendicular to the propagation of the ultrasound beam. A single element transducer (1 MHz, 4.40 MPa peak-to-peak at the focus of 63 mm; NDT A392S, Olympus, Shinjuku, Tokyo, Japan) was submerged in deionized water to deliver focused ultrasound pulses near the DBS device.

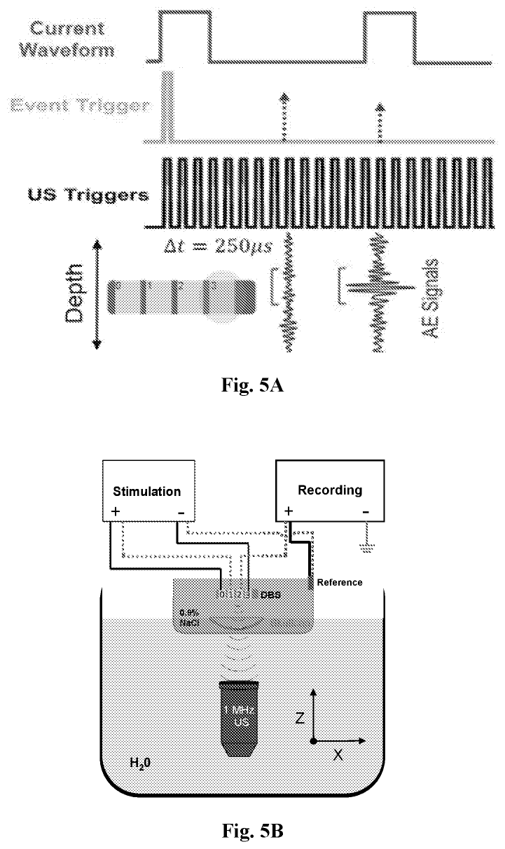

FIG. 5A depicts the timing diagram for acquiring AE signals along one line (depth, z) with the center of the transducer at coordinates x, y. Time-varying pulses of injected current (3V, 200 Hz, 1 ms rectangular pulse) were delivered from a function generator (33220A, Agilent, Santa Clara, Calif., USA) to either two DBS contacts (dipole configuration) or one DBS contact and a distant stainless-steel ground electrode (monopole configuration). The stimulation waveform was similar to those used clinically. A burst of ultrasound pulses was focused near the stimulating contacts at a pulse repetition frequency of 4 kHz simultaneous with the injected current. A distant reference electrode (dipole configuration) or adjacent contact on the DBS (monopole configuration) was used to record the AE interaction signal (FIG. 5B). For dipole imaging, 12 bipolar configurations were theoretically possible when considering all leads and polarities; however, the inventors implemented only three unique combinations (3-2, 3-1, and 3-0) to demonstrate spatial selectivity of AEI.

The AE signal passed through a custom 10 MHz differential amplifier with analog filtering (˜3 dB frequency cutoffs at 0.2 and 2 MHz) and a gain of 40 dB. The signal was then digitized at 20 MHz on an eight channel, 12-bit NI PXI-5105 acquisition card (National Instruments, Austin, Tex., USA). The injected current was measured across a 1 Ohm resistor in series with the stimulating electrodes, amplified 10× by a differential amplifier (PA1855A, Teledyne Lecroy, Chestnut Ridge, N.Y., USA) and digitized at 20 kHz on a NI PXI-6289 acquisition card (National Instruments, Austin, Tex., USA). AE M-Mode images (depth vs. t_{slow}) were generated with the ultrasound beam at one location along the lateral and elevational axes. The ultrasound transducer was also mechanically scanned in the lateral plane at step sizes of 0.33 mm to form cross sectional 2D AE images (lateral vs depth) and movies over time related to the location, direction and amplitude of the local current densities. Standard pulse echo ultrasound was simultaneously captured on a pulser-receiver (5077PR, Olympus, Shinjuku, Tokyo, Japan) and digitized on the same NI PXI-5105 acquisition card at 20 MHz to determine the position and orientation of the tip of the DBS device co-registered to the AE signal.

C. Acoustoelectric Imaging through Human Skullcap

A skullcap was placed upside down in a chamber filled with 0.9% saline, 36 mm below the DBS contacts and 25 mm above the ultrasound transducer such that the propagating beam passed through the 6.0-mm thick parietal bone 2 cm lateral from the coronal suture. The AE signals were averaged up to 50 times per location along the lateral axis for the monopole and dipole images and for calculating sensitivity and SNR. A capsule hydrophone (HGL-0200, Onda Corporation, Sunnyvale, Calif., USA) was used to calibrate the pressure field with and without the skullcap.

D. Post Processing and Analysis

AE signals were bandpass filtered with −3 dB cutoffs at 0.5 and 1.5 MHz (along ultrasound propagation time; t_{fast}) and 100 Hz and 1000 Hz (along current waveform time; t_{slow}). The real AE signals were Hilbert transformed and basebanded to form the complex envelope. Whereas the absolute value of the complex envelope determined the magnitude of the local current densities, the sign of the complex envelope determined polarity. AE images are displayed on hot/cold color maps to indicate the magnitude (intensity) and polarity (color) of the local current densities. Co-registered pulse echo B-mode images are displayed in grayscale.

The signal-to-noise ratio (SNR) was calculated for different amounts of trial averaging (from 1 to 64) to assess background noise and variability. SNR was calculated from the peak of the envelope of the AE signal divided by the maximum voltage in a region devoid of signal. The SNR in dB was then calculated as 20*log₁₀(signal/noise).

Sensitivity in μV/mA was determined by calculating the slope between the AE signal and injected current. This was then normalized to peak-to-peak pressure at the ultrasound focus to determine sensitivity in μV/(MPa*mA). The detection threshold for determining minimum detectable current was defined as the mean+twice the standard deviation of the noise. Six trials using a 200 Hz stimulating sine wave was passed through the DBS device in a monopole configuration for these calculations. Ultrasound pulsed at 4 kHz focused at the monopole provided a temporal resolution of 250 μs for the AE amplitude measurements. Peak negative pressures were less than 1.9 MPa for all experiments (i.e., the mechanical index was <1.9, the FDA safety limit for diagnostic imaging).

The spatial extent of monopoles generated by the DBS device were calculated at full-width half-maximum (FWHM) in both axial and lateral directions (n=12 for monopoles, 9 for dipoles). The center of each stimulating contact was estimated from the peak of the local current densities. The location of the DBS device in physical space was determined by the pulse echo (PE) signal (two-way ultrasound travel), which was co-registered and superimposed with AEI (one-way ultrasound travel). The expected distance between the peaks was expected to close to the 2-mm pitch between consecutive DBS contacts.

2.2 Results

A. AEI of Monopole Current Pulses

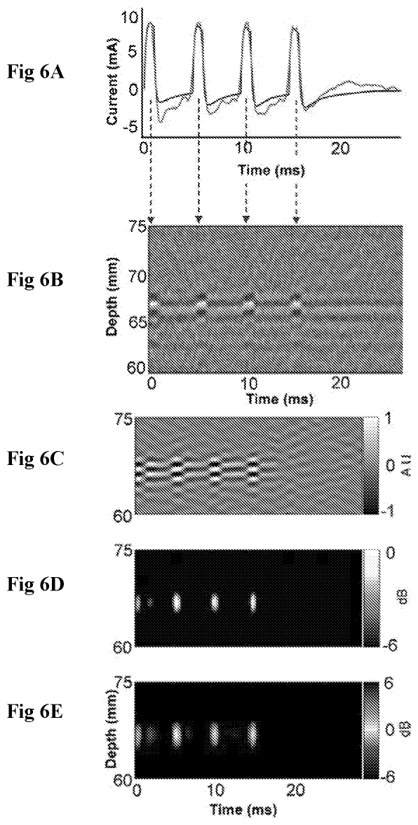

The AE M-Mode image (FIG. 6B) displays bright peaks at the time of the current pulses and depth corresponding to contact 3 on the DBS device. A slight deflection below baseline is also observed (as indicated by the vertical dashed arrows), consistent with the current waveform measured simultaneously. FIG. 6A depicts the AE M-Mode signal (gray line) at a single depth (67.5 mm=depth of the DBS device), which is superimposed and highly correlated (R²=0.886) with the injected current waveform (black line), illustrating the fast temporal resolution of AEI.

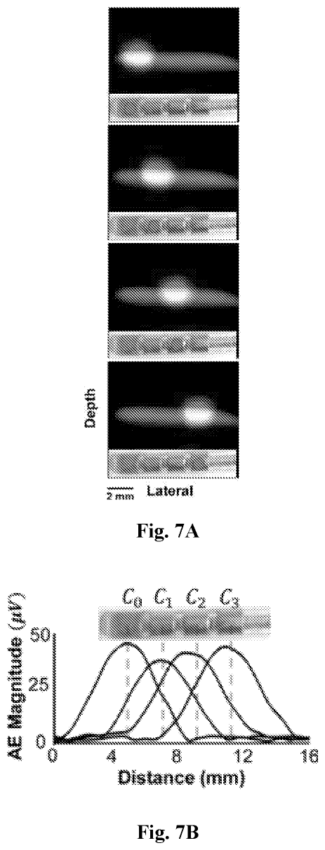

Next, the ultrasound beam was scanned along the DBS leads to form 3D (lateral, depth and time) images depicting time-varying current densities for monopoles at each of the four contacts. FIG. 7A illustrates the PE image of the DBS with the superimposed AE image for each monopole configuration. The peak amplitude (mean±std) of the AE signals from the monopoles were 42.5±4.5 μV. The dimensions of the AE signals in the lateral and axial directions were 3.67±0.18 mm and 3.11±0.21 mm, respectively. The coordinates of the peak signal along the lateral axis were also calculated for each monopole and were within 0.41 mm of the actual location of the stimulating contact estimated by PE. The line profiles in FIG. 7B compare the AE images with the physical location of the contacts.

B. AEI of Dipole Current Pulses

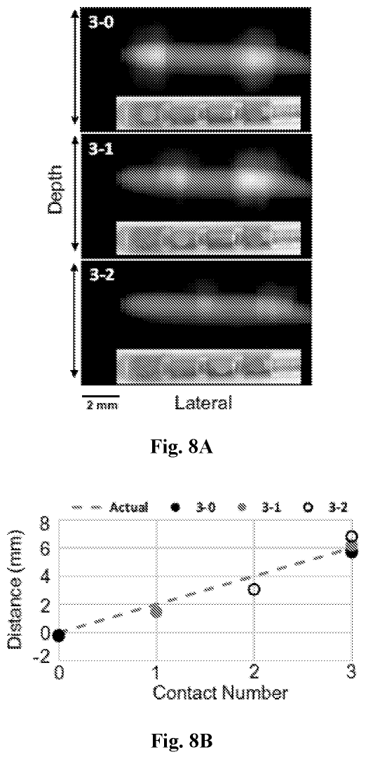

AEI was also used to map dipoles generated by the DBS electrodes to examine effects of polarity on the sensitivity and accuracy of AEI. The anode was fixed (contact 3) for all scans, while the cathode was shifted among the remaining contacts. Three dimensional movies were generated for these 3 dipole configurations (see supplemental movie 2 for the 3-1 dipole). The 2D images taken at the peak of the AE signal (FIG. 8A) clearly illustrate the shift in the location of the cathode (blue). The peak magnitude, SNR and width of each pole were measured for the bipolar stimulating configurations (summarized in Table 2 below). The 3-1 and 3-0 dipoles had comparable peak amplitudes, but slightly less than the monopole configuration. The 3-2 configuration had a lower amplitude for both poles. Additionally, the lateral extent of each pole detected by AEI decreased from configuration 3-0 to 3-2, most likely due to volume integration over the ultrasound focus (see discussion).

TABLE 2
AEI quantification using different dipole configurations.

| DBS Contacts (A-C) | 3-0 | 3-1 | 3-2 |
|---|---|---|---|
| Anode AE Peak (μV) | 32.8 | 35.6 | 21.5 |
| Cathode AE Peak (μV) | 30.1 | 31.0 | 23.0 |
| Anode SNR (dB) | 19.4 | 20.1 | 15.8 |
| Cathode SNR (dB) | 18.7 | 19.0 | 16.4 |
| Anode Width (mm) | 3.27 | 3.57 | 2.13 |
| Cathode Width (mm) | 3.27 | 3.17 | 1.98 |

Peak refers to peak of AE signal envelope. Width was measured at −6 dB. SNR was calculated with an injected current of 11.5 mA.
Mechanical Index = 1.7.
A = Anode; C = Cathode.

The centers of the anode from AEI for the 3-0 and 3-1 configurations were 5.82 mm and 6.11 mm, which were close to the expected position of 6 mm (FIG. 8B). In contrast, the center of the anode determined by AEI using the 3-2 configuration was 6.81 mm. Finally, the distance between the cathode and anode was measured for each configuration and subtracted from the actual distance of the center of the stimulating contacts. The differences were −0.06 mm (narrower), +0.75 mm (wider), and +1.74 mm for the 3-0, 3-1, and 3-2 configurations, respectively.

C. AEI Sensitivity and SNR

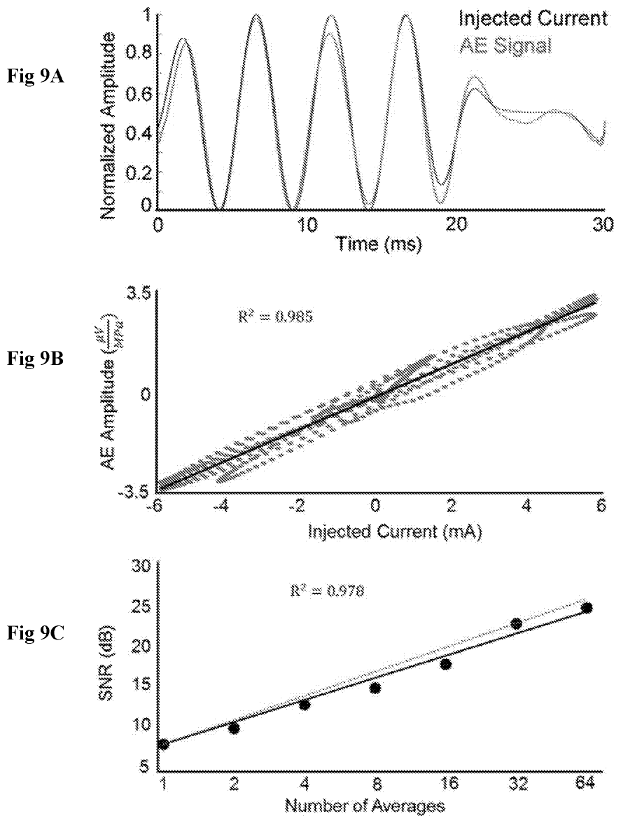

The sensitivity determined from the 200 Hz sine wave stimulation was 0.52 μV/(MPa*mA) with R²=0.985 (see FIGS. 9A and 9B). Based on a detection threshold of 0.91 μV at 1 MPa, the smallest detectable current was 1.75 mA.

A plot of SNR vs number of repeated stimulations (N) revealed the effect of averaging for detecting current generated by the DBS device (FIG. 9C). With no averaging, SNR was 8 dB and increased logarithmically (base 2) with the number of averages (R²=0.978) with a slope of 2.74 dB/octave, reaching 25 dB at 64 averages. This pattern followed closely the assumption of independent background noise between trials (\sqrt{N}).

D. Transcranial AEI of DBS Current Densities

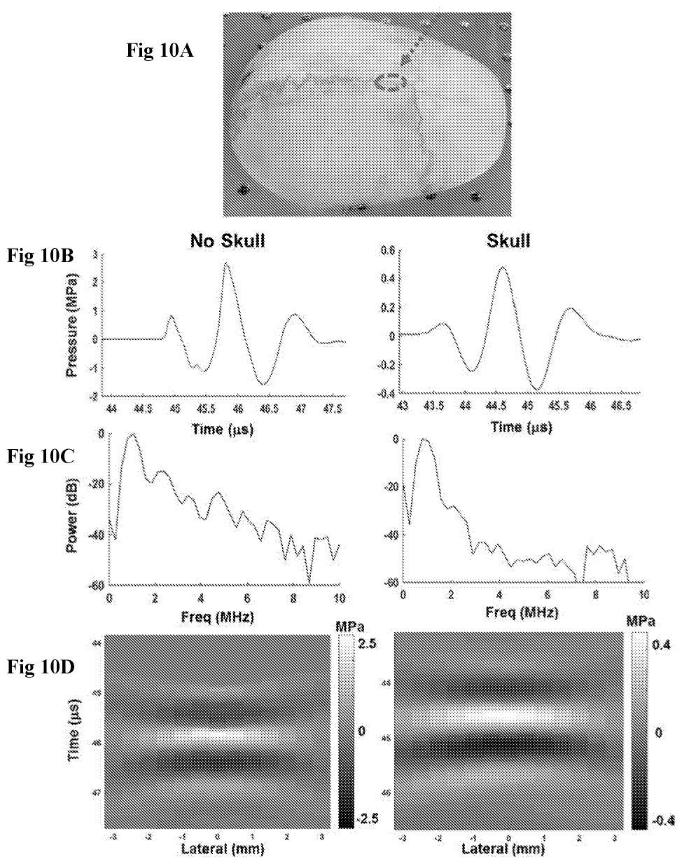

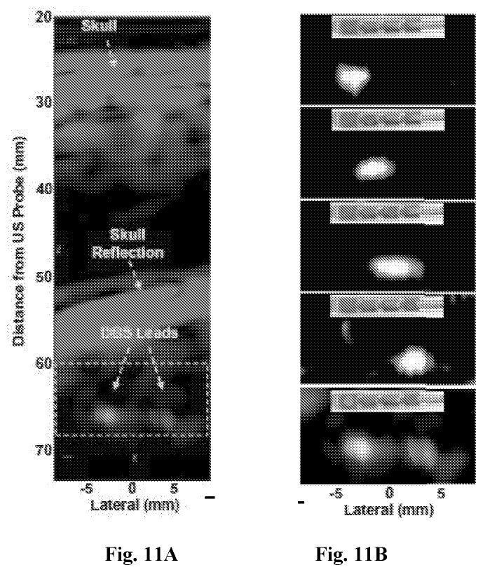

Peak pressure at the depth of the DBS electrode decreased from 4.40 MPa to 0.868 MPa (80.3% attenuation) after the ultrasound beam passed through the skull (at normal incidence) corresponding to an attenuation coefficient of 2.74 Np/m. The beam width (lateral FWHM) was 21.3% larger after propagating through the skull with minimal change to the axial (transverse normal) focus (FIGS. 10A-10D). The speed of sound of the 6.0-mm thick bone was calculated at 3157 m/s based on the time delay through the skull of −2.11 μs.

Although the tip of the DBS was discernible on PE images through the skullcap, these images were low quality due to skull reflections and two-way attenuation of the acoustic wave (FIG. 11A). The AE signal, on the other hand, remained fully detectable and characterizable (FIG. 11B). The lateral and axial dimensions of the monopole through skull were 4.71 and 3.24 mm, respectively. The peak locations of the monopoles generated at contacts 1, 2, and 3 were 1.56, 3.81, and 6.15 mm away from the peak of the contact 0 monopole. Compared to the expected distances of 2, 4, and 6 mm, the error in relative accuracy of the monopole locations was ≤0.44 mm. The distance between each pole in the 0-3 dipole configuration was 6.14 mm, compared to the expected 6 mm (FIG. 11B—bottom). Sensitivity for detecting the AE signal was 0.56 μV/(MPa*mA) with R²=0.971. At the detection threshold of 0.91 μV at 1 MPa, the corresponding threshold for current detection was 1.65 mA.

2.3 Discussion

A. Resolution and Accuracy for Identifying Current Sources Generated by DBS

AEI provided quantitative maps of local current densities generated by a DBS device using stimulation parameters resembling those used in patients. At 1 MHz, AEI was able to spatially resolve monopole and dipole sources generated by DBS with sub-mm accuracy and a detection threshold below 0.40 mA at safe ultrasound pressures. This was verified by systematically switching the stimulation contacts and scanning the ultrasound transducer to form co-registered 3D AE and pulse echo ultrasound images. AEI was also able to temporally resolve the peak magnitude, SNR, and pulse width at with 250 μs sampling limited only by the ultrasound pulse repetition rate of the transmitter. The integration of AEI with pulse echo ultrasound could help quantify spatial patterns of current flow and enable estimating volumes of tissue activated during therapeutic DBS. Such empirical feedback in the human brain would help validate and optimize computational models of DBS while also enhancing the understanding of mechanisms underlying effective DBS. It may also be possible to register current density maps from AEI with structural MR images to help guide placement of the DBS device with sub-mm accuracy. AEI was able to accurately resolve the direction and amplitude of dipoles generated by the DBS device. The decrease in amplitude of the source and sink (illustrated in FIG. 8A) as the stimulating contacts merged suggests that the two poles begin to cancel due to the volume integration of the ultrasound beam focus (˜3.0 mm) according to equation (1). This effect could be reduced by employing a high frequency transducer to improve spatial resolution for AEI.

B. Noninvasive AEI Through Human Skull

The effects of AEI through the skullcap were consistent with ultrasound attenuation and aberrations and aberrations through bone. The SNR of the AE signal when focusing through the skull decreased on average by 13.8 dB. This matched closely with the expected drop in SNR of 14.3 dB given the 80.3% attenuation in pressure. The width and height of the monopoles measured by AEI increased 28.3% and 5%, respectively, through skull, which was consistent with the corresponding increase in the ultrasound focal size (Table 3 below). The resulting attenuation coefficient of 2.74 Np/cm is slightly larger than that measured by Ammi et al., who calculated a mean attenuation coefficient for human skull of 2.00 Np/cm when using a 1.03 MHz unfocused ultrasound transducer. This difference may be explained by different properties or condition of the skull. The measured speed of sound in the skull of 3157 m/s is within range of other studies that report a range between 2800 and 3300 m/s. After normalizing to pressure, the sensitivity for detecting AE signals increased only slightly (7.6%) through the skullcap (Table 3 below). Because the skull did not increase background noise on the recording electrodes, the threshold for current detection was primarily affected by the reduction in pressure.

TABLE 3
AEI quantification with and without human skullcap.

| | No Skull | Skull |
|---|---|---|
| Pk-Pk Pressure (MPa) | 4.40 | 0.868 |
| Beam Width (mm) | 3.34 | 4.05 |
| Beam Height (mm) | 2.34 | 2.38 |
| Monopole Width (mm) | 3.67 ± 0.18 | 4.71 ± 0.41 |
| Monopole Height (mm) | 3.11 ± 0.21 | 3.24 ± 0.25 |
| Sensitivity (μV/MPa/mA) | 0.52 ± 0.07 | 0.56 ± 0.10 |
| SNR (dB) | 24.7 ± 1.6 | 10.9 ± 1.1 |

Width (lateral) and height (transverse normal) were calculated at −6 dB. DBS lead dimensions were 1.5 mm (width) and 1.27 mm (height). SNR was calculated at 11.5 mA. Mechanical indices at US focus were 0.386 (skull) and 1.70 (no skull). Monopole data represents the average for all four lead configurations.

The significant reduction in the PE signal paired with focal aberrations explain the degradation of the pulse echo images of the DBS through the skullcap. A phased-array ultrasound transducer combined with correction algorithms (e.g., forward beamforming or time reversal) should help reduce distortion of the ultrasound beam through bone. The reflection artifact on PE due to the skull (see FIG. 11A) introduces another potential complication for AEI. In any practical clinical setting, however, the ultrasound transducer would be directly coupled to the skin over the skull such that the distance to the skull interface would be small, resulting in a reflection that is far removed from the target location of the implanted DBS electrode.

Ultimately, as a noninvasive modality for imaging the human brain, any post-operative AEI performed through the skull would benefit from the highest ultrasound frequency possible through skull. Transcranial Doppler (TCD) arrays operate near 2 MHz and are commonly used to deliver ultrasound through the temporal window. An investigation of the interaction between ultrasound and the human temporal bone reported a mean attenuation coefficient of 4.76 Np/cm at 2.00 MHz. Given a 2.5 mm thick temporal window, the estimated attenuation of the ultrasound beam would be 69.6% and less than the 80.3% attenuation, which still maintained an SNR>10 dB for AEI. Therefore, it seems possible that AEI could be performed through the temporal window for targeting deep brain structures and mapping local current densities near the DBS with sub-mm precision. In this fashion, it would also be possible to fuse real-time AEI and pulse echo ultrasound images of the DBS implant onto anatomical MR images for lead localization or for guiding placement during surgery.

One common complication in TCD is known as temporal bone window insufficiency, where the thickness of the skull at the temporal window is too large, preventing effective imaging. This occurs based on normal variations in bone thickness between people and is estimated to reduce or prevent TCD in 29% of the global population. Because AEI only requires one-way propagation of ultrasound, there is considerably less attenuation through the skull than for TCD or PE imaging. Although deep brain structures are accessible through temporal windows, it may also be possible to deliver ultrasound directly through thicker part of the skull for AEI. In fact, the inventors have recently designed a 0.6 MHz planar matrix array and demonstrated feasibility in a human head phantom using artificial current sources for 4D AEI through thick skull. However, with a resolution >3 mm, this low frequency array may not be ideal for selective imaging of contacts on a DBS device. Regardless, most common DBS applications (e.g., PD, ET, Dystonia) target deep brain structures accessible through the temporal window.

C. Current Steering and Orientation-specific DBS

Accurate placement of the DBS electrodes is key to its success in alleviating motor symptoms and minimizing side-effects. Intraoperative MRI guided and CT techniques have been developed to enhance the accuracy of electrode placement from pre-operative anatomical MRI. However, errors in both prediction and placement still occur, limiting the overall success of DBS. For example, a DBS lead implanted in the subthalamic nucleus that lies too close to its lateral boundary can also excite the internal capsule due to its laterally symmetrical field shaping. Omni-directional DBS devices with current steering capability, such as the Sapiens SureStim™, and Abbot Infinity™, have recently been introduced for clinical use. These newer DBS devices allow for current to be steered toward a select side of the device, which may help overcome misplacement during surgery by maintaining optimal volumetric stimulation. AEI presents an elegant way to verify and troubleshoot the complex electric field patterns produced by a steerable DBS device during or after surgery without the need for additional or invasive electrodes.

Precise current steering and field shaping is one approach for optimizing DBS parameters through selective excitation of volumes of tissue, which would allow for circuit selective excitation and strict control of the second spatial derivative of extracellular electric fields driving neuronal excitation. Several studies have demonstrated that DBS electrode designs can exploit the orientation dependence of neurons. AEI may be able to provide access to in-vivo data from DBS in humans and enable feedback regarding the population of neurons and circuits responsible for optimal relief of PD symptoms. In this manner, AEI can help guide and validate models of therapeutic DBS for PD by providing real-time spatiotemporal feedback of current densities as they are steered around the implant.

2.4 Conclusion

This disclosure demonstrates feasibility of AEI for selectively mapping the magnitude and polarity of current source densities generated by a clinical DBS device with high resolution, sub-mm and sub-ms accuracy, and detection thresholds below 1.75 mA at 1 MPa. Because most deep brain structures are readily accessible with ultrasound through the temporal window, high resolution AEI may be possible for a variety of applications relevant to DBS. As a clinical tool, AEI could help guide placement of an implant during surgery, optimize stimulation parameters, or monitor its performance in patients. Finally, as a noninvasive modality for mapping local current densities, AEI would provide quantitative empirical data in the human brain for validating computational models and improving the understanding of therapeutic DBS.

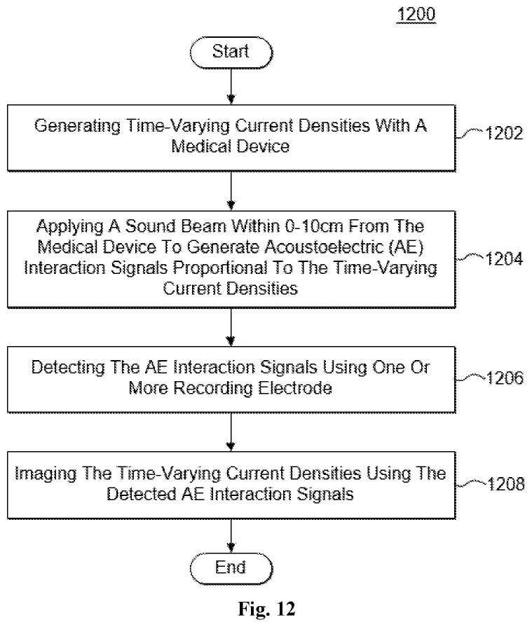

Embodiments are directed to a method for acoustoelectrically imaging, within a body part, time-varying current densities generated by a medical device. FIG. 12 is a flowchart illustrating an embodiment of a method 1200 for acoustoelectrically imaging, within a body part, time-varying current densities generated by a medical device, in accordance with an embodiment. The method 1200 comprises: generating time-varying current densities with a medical device (block 1202); applying a sound beam within 0-10 cm from the medical device to generate acoustoelectric (AE) interaction signals proportional to the time-varying current densities (block 1204); detecting the AE interaction signals using one or more recording electrode (block 1206); and imaging the time-varying current densities using the detected AE interaction signals (block 1208). Embodiments of the method 1200 are capable of acoustoelectrically imaging time-varying current densities in the brain or other body parts. The AE interaction signals are detected on the one or more electrode. The detected AE interaction signals are then passed to a preamplifier (with gain and bandpass filtering), an analog to digital (A2D) converter, optional post processing, and ultimately to a display for the imaging.

In an embodiment, the sound beam comprises an ultrasound beam. The AE interaction signals may also be proportional to a focal pressure from the ultrasound beam.

In an embodiment, the medical device comprises a deep brain stimulator (DBS) and the body part is the brain. The imaged time-varying current densities are located within the brain and are imaged through the parietal bone.

In an embodiment, the imaging is used in an application selected from the group consisting of guiding placement of the medical device during surgery, monitoring performance of the medical device during a check-up, performing an accurate calibration of the medical device, and a combination thereof.

In an embodiment, the one or more recording electrode is placed on a surface of the body part to detect the AE interaction signals.

In an embodiment, the one or more recording electrode is part of or attached to the medical device.

In an embodiment, the medical device comprises a pacemaker, transcranial magnetic stimulation (TMS), vagal nerve stimulator, or transcutaneous electrical nerve stimulation (TENS) device.

Embodiments are also directed to an acoustoelectric imaging system that acoustoelectrically images, within a body part, time-varying current densities generated by a medical device. In an embodiment, the acoustoelectric imaging system comprises: a medical device that generates time-varying current densities; a sound beam system that applies a sound beam within 0-10 cm from the medical device to generate AE interaction signals proportional to the time-varying current densities; one or more recording electrode that detects the AE interaction signals; and a current density imaging system that images the time-varying current densities using the detected AE interaction signals. Embodiments of the system are capable of acoustoelectrically imaging time-varying current densities in the brain or other body parts. The AE interaction signals are detected on the one or more electrode. The detected AE interaction signals are then passed to a preamplifier (with gain and bandpass filtering), an analog to digital (A2D) converter, optional post processing, and ultimately to a display for the imaging.

In an embodiment, the sound beam system comprises an ultrasound beam system, and wherein the sound beam comprises an ultrasound beam. The AE interaction signals may also be proportional to a focal pressure from the ultrasound beam.

In an embodiment, the medical device comprises a DBS and the body part is the brain. The imaged time-varying current densities are located within the brain and are imaged by the current density imaging system through the parietal bone.

In an embodiment, the imaged time-varying current densities are used in an application selected from the group consisting of guiding placement of the medical device during surgery, monitoring performance of the medical device during a check-up, performing an accurate calibration of the medical device, and a combination thereof.

In an embodiment, the one or more recording electrode is placed on a surface of the body part to detect the AE interaction signals.

In an embodiment, the one or more recording electrode is part of or attached to the medical device.

In an embodiment, the medical device comprises a pacemaker, transcranial magnetic stimulation (TMS), vagal nerve stimulator, or TENS device.

Although embodiments are described above with reference to systems and methods for acoustoelectrically imaging, within the brain, time-varying current densities generated by a medical device such as a DBS, the systems and methods may alternatively or additionally be applied to other parts of the body. Such alternatives are considered to be within the spirit and scope of the present invention, and may therefore utilize the advantages of the configurations and embodiments described above.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A method for acoustoelectrically imaging, within a body part, time-varying current densities generated by a medical device, the method comprising:
generating time-varying current densities with a medical device;
applying a sound beam within 0-10 cm from the medical device to generate acoustoelectric (AE) interaction signals proportional to the time-varying current densities;
detecting the AE interaction signals using one or more recording electrode; and
imaging the time-varying current densities using the detected AE interaction signals.

2. The method of claim 1, wherein the sound beam comprises an ultrasound beam.

3. The method of claim 2, wherein the AE interaction signals are also proportional to a focal pressure from the ultrasound beam.

4. The method of claim 1, wherein the medical device comprises a deep brain stimulator (DBS).

5. The method of claim 4, wherein the body part is the brain, and wherein the imaged time-varying current densities are located within the brain and are imaged through the parietal bone.

6. The method of claim 1, wherein the imaging is used in an application selected from the group consisting of guiding placement of the medical device during surgery, monitoring performance of the medical device during a check-up, performing an accurate calibration of the medical device, and a combination thereof.

7. The method of claim 1, wherein the one or more recording electrode is placed on a surface of the body part to detect the AE interaction signals.

8. The method of claim 1, wherein the one or more recording electrode is part of or attached to the medical device.

9. The method of claim 1, wherein the medical device comprises a pacemaker, transcranial magnetic stimulation (TMS), vagal nerve stimulator, or transcutaneous electrical nerve stimulation (TENS) device.

10. An acoustoelectric imaging system that acoustoelectrically images, within a body part, time-varying current densities generated by a medical device, the acoustoelectric imaging system comprising:
a medical device that generates time-varying current densities;
a sound beam system that applies a sound beam within 0-10 cm from the medical device to generate acoustoelectric (AE) interaction signals proportional to the time-varying current densities;
one or more recording electrode that detects the AE interaction signals; and
a current density imaging system that images the time-varying current densities using the detected AE interaction signals.

11. The acoustoelectric imaging system of claim 10, wherein the sound beam system comprises an ultrasound beam system, and wherein the sound beam comprises an ultrasound beam.

12. The acoustoelectric imaging system of claim 11, wherein the AE interaction signals are also proportional to a focal pressure from the ultrasound beam.

13. The acoustoelectric imaging system of claim 10, wherein the medical device comprises a deep brain stimulator (DBS).

14. The acoustoelectric imaging system of claim 13, wherein the body part is the brain, and wherein the imaged time-varying current densities are located within the brain and are imaged by the current density imaging system through the parietal bone.

15. The acoustoelectric imaging system of claim 10, wherein the imaged time-varying current densities are used in an application selected from the group consisting of guiding placement of the medical device during surgery, monitoring performance of the medical device during a check-up, performing an accurate calibration of the medical device, and a combination thereof.

16. The acoustoelectric imaging system of claim 10, wherein the one or more recording electrode is placed on a surface of the body part to detect the AE interaction signals.

17. The acoustoelectric imaging system of claim 10, wherein the one or more recording electrode is part of or attached to the medical device.

18. The acoustoelectric imaging system of claim 10, wherein the medical device comprises a pacemaker, transcranial magnetic stimulation (TMS), vagal nerve stimulator, or transcutaneous electrical nerve stimulation (TENS) device.
